(19) United States
(12) Patent Application Publication
(10) Pub. No.: US 2022/0211263 A1
(43) Pub. Date: Jul. 7, 2022

(54) MULTI-PIECE ACCESS PORT IMAGING SYSTEMS

(71) Applicant: Axcess Instruments Inc., Tyler, TX (US)

(72) Inventors: Michael J. Norton, Tyler, TX (US); Noel D. Ischy, Tyler, TX (US)

(73) Assignee: Axcess Instruments Inc., Tyler, TX (US)

(21) Appl. No.: 17/702,193

(22) Filed: Mar. 23, 2022

Related U.S. Application Data
(60) Provisional application No. 63/165,045, filed on Mar. 23, 2021.

(51) Int. Cl. A61B 1/32; A61B 1/00

(52) U.S. Cl. CPC A61B 1/32; A61B 1/00071; A61B 1/00009

(57) ABSTRACT

An access port system can include a port body configured to be inserted into an incision. The port body can define an imaging assembly opening from a proximal side to a distal side thereof. The system can include an imaging assembly configured to pass through the imaging assembly opening to allow imaging of an interior portion of a patient body.

### CROSS-REFERENCE TO RELATED APPLICATIONS

This application claims priority to and the benefit of U.S. Provisional Application No. 63/165,045, filed Mar. 23, 2021, the entire contents of which are herein incorporated by reference in their entirety.

### FIELD

This disclosure relates to access ports, for example.

### BACKGROUND

Conventional methods and systems in the laparoscopic and access port arts have generally been considered satisfactory for their intended purpose. However, there is still a need in the art for improved systems. The present disclosure provides a solution for this need.

### SUMMARY

An access port system can include a port body configured to be inserted into an incision. The port body can define an imaging assembly opening from a proximal side to a distal side thereof. The system can include an imaging assembly configured to pass through the imaging assembly opening to allow imaging of an interior portion of a patient body.

The port body can be flexible and a housing of the imaging assembly can be rigid. The port body can also define an instrument channel from the proximal side thereof to the distal side to allow an instrument pass through to perform a medical procedure.

The port body can include one or more anchors extending from the port body. The one or more anchors can include a heal anchor adjacent the distal side of the port body. The one or more anchors can include a toe anchor disposed on an opposite side of the port body relative to the heal anchor and proximal of the heal anchor.

The imaging assembly can include a radial portion configured to house an imaging device disposed therein and a leg portion extending proximally from the radial portion. The imaging assembly can include a boot shape, for example.

The leg portion can be angled at a right angle to the radial portion. The imaging assembly can include an imaging device located in the radial portion at a radially outward position thereof.

The port body can include an instrument channel defined therethrough from a proximal side to a distal side thereof. The port body can include an insufflation port defined therethrough from a proximal side to a distal side thereof. In certain embodiments, the port body is made of silicone.

The port body and the imaging assembly can be configured such that imaging assembly does not rotate within the port body when inserted into the port body. The port body can define a window that seals to the radial portion of the imaging assembly.

The port body can include a swept back shape. An imaging device housing of the imaging device can include a swept back shape. For example, the imaging device housing can include an oval cross-sectional shape.

In certain embodiments, the imaging assembly can be a straight member having an angled distal face. An imaging device can be disposed at the angled distal face to provide an angled view when inserted through the port body.

In certain embodiments, the system can include an image processing module configured to allow digital movement of an image in situ. Any other suitable image processing is contemplated herein.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a top down plan view of an embodiment of a system in accordance with this disclosure;

FIG. 2 is a cross-sectional view of the embodiment of FIG. 1, the section being taken along line 2-2 as shown in FIG. 1;

FIG. 3 is a perspective view of the embodiment of FIG. 1;

FIG. 4 is a perspective view of the embodiment of FIG. 1;

FIG. 5 is a side elevation view of the embodiment of FIG. 1;

FIG. 6 is a bottom up plan view of the embodiment of FIG. 1;

FIG. 7 is a top down perspective view of the embodiment of FIG. 1, showing the radial extension in a rotated position;

FIG. 8 is a bottom up view of the embodiment of FIG. 7;

FIG. 9 is a front elevation view of the embodiment of FIG. 7;

FIG. 10 is a side elevation view of the embodiment of FIG. 1, showing a port body being moved upwardly relative to an imaging assembly;

FIG. 11 is a perspective view of an imaging assembly of the embodiment of FIG. 1, shown in isolation;

FIG. 12 is a top down plan view of a port body of the embodiment of FIG. 1, shown in isolation;

FIG. 13 is a side schematic view of the embodiment of FIG. 12, showing internal channels in phantom;

FIG. 14 is a perspective view of the embodiment of FIG. 12;

FIG. 15 is a perspective view of the embodiment of FIG. 12;

FIG. 16 is a perspective view of another embodiment of an imaging assembly in accordance with this disclosure;

FIG. 17 is a bottom perspective view of the embodiment of FIG. 16;

FIG. 18 is a cross-sectional view of the embodiment of FIG. 16;

FIG. 19 is a perspective view of another embodiment of a port body in accordance with this disclosure, shown without an instrument channel;

FIG. 20 is a perspective view of the embodiment of FIG. 19;

FIG. 21 is a perspective view of another embodiment of a port body in accordance with this disclosure, shown without an instrument channel;

FIG. 22 is a perspective view of the embodiment of FIG. 19;

FIG. 23 is a perspective view of another embodiment of an access port system in accordance with this disclosure;

FIG. 24 is a left side elevation view of the embodiment of FIG. 23;

FIG. 25 is a top down plan view of the embodiment of FIG. 23;

FIG. 26 is a bottom up plan view of the embodiment of FIG. 23;

FIG. 27 is a right side elevation view of the embodiment of FIG. 23;

FIG. 28 is a front side elevation view of the embodiment of FIG. 23;

FIG. 29 is a cross-sectional view of the embodiment of FIG. 23, sectioned along line 29-29 in FIG. 28;

FIG. 30 is a rear side elevation view of the embodiment of FIG. 23;

FIG. 31 is a cross-sectional view of the embodiment of FIG. 23, sectioned along line 31-31 in FIG. 30;

FIG. 32 is a perspective view of the embodiment of FIG. 23;

FIG. 33 is another perspective view of the embodiment of FIG. 23;

FIG. 34 is a right side elevation view of an embodiment of a port body of the embodiment of FIG. 23, shown in isolation;

FIG. 35 is a front elevation view of the embodiment of FIG. 34;

FIG. 36 is a top down plan view of the embodiment of FIG. 34;

FIG. 37 is a perspective view of the embodiment of FIG. 34;

FIG. 38 is a rear elevation view of the embodiment of FIG. 34;

FIG. 39 is a bottom up plan view of the embodiment of FIG. 34;

FIG. 40 is a front elevation view of the embodiment of FIG. 34, shown enlarged;

FIG. 41 is a cross-sectional view of the embodiment of FIG. 34, sectioned along line 40-40 in FIG. 40, showing an embodiment of an instrument port and a imaging assembly port;

FIG. 42 is a front elevation view of the embodiment of FIG. 34, shown enlarged;

FIG. 43 is a cross-sectional view of the embodiment of FIG. 34, sectioned along line 43-43 in FIG. 40, showing an embodiment of an insufflation port;

FIG. 44 is a perspective view of an embodiment of an imaging assembly housing of the embodiment of FIG. 23, shown in isolation;

FIG. 45 is a right side elevation view of the embodiment of FIG. 44;

FIG. 46 is a cross-sectional view of the embodiment of FIG. 45, sectioned along line 46-46 in FIG. 40;

FIG. 47 is a left side elevation view of the embodiment of FIG. 44;

FIG. 48 is a cross-sectional view of the embodiment of FIG. 44, sectioned along line 48-48 in FIG. 40;

FIG. 49 is a bottom up plan view of the embodiment of FIG. 44;

FIG. 50 is a top down plan view of the embodiment of FIG. 44;

FIG. 51 is a left side elevation view of an embodiment of a right half of the embodiment of FIG. 44;

FIG. 52 is a right side elevation view of an embodiment of a left half of the embodiment of FIG. 44;

FIG. 53 is a front elevation view of the embodiment of FIG. 44; and

FIG. 54 is a front elevation view of the embodiment of FIG. 44.

### DETAILED DESCRIPTION

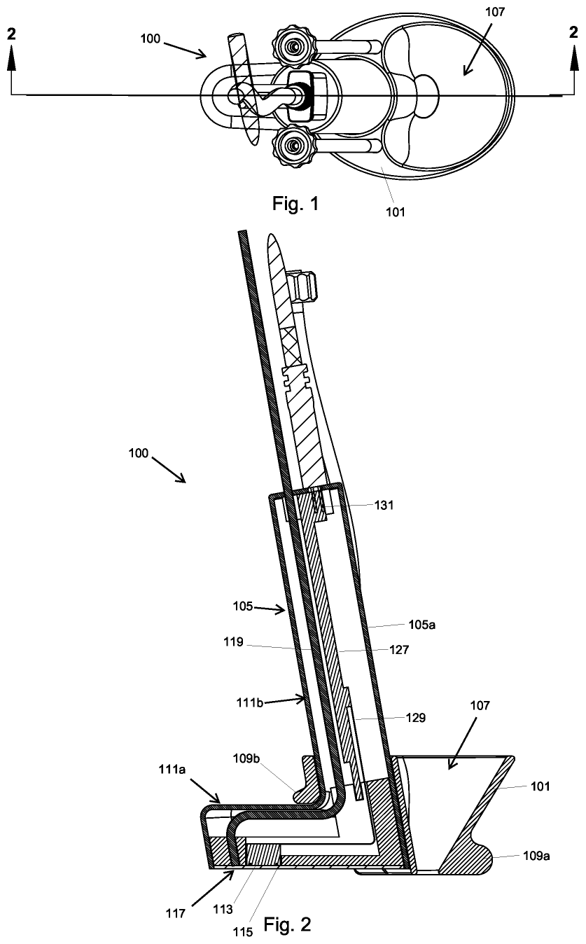

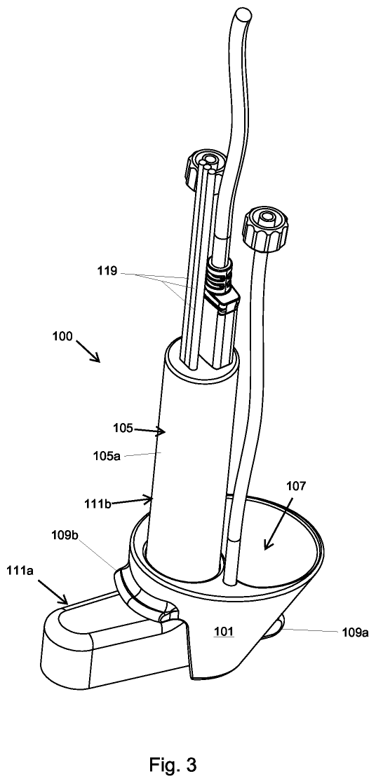

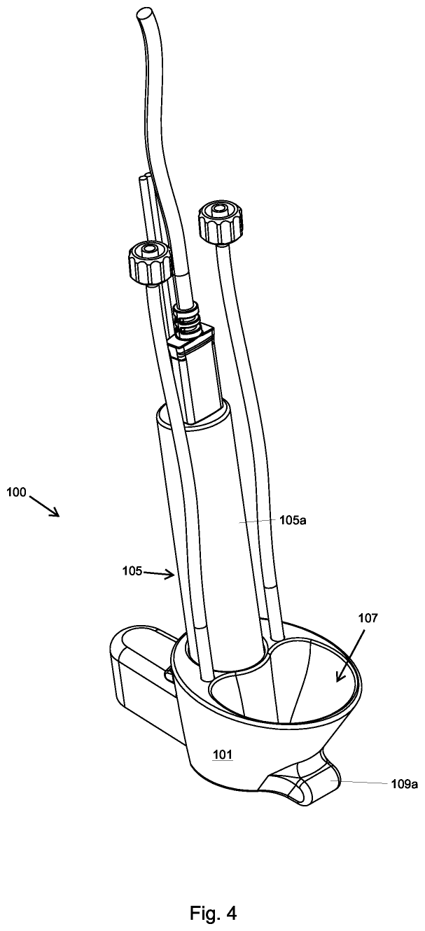

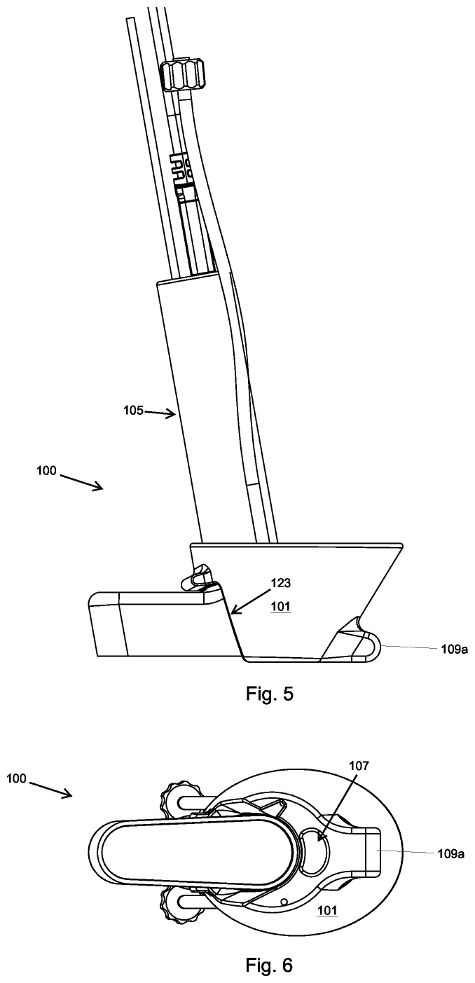

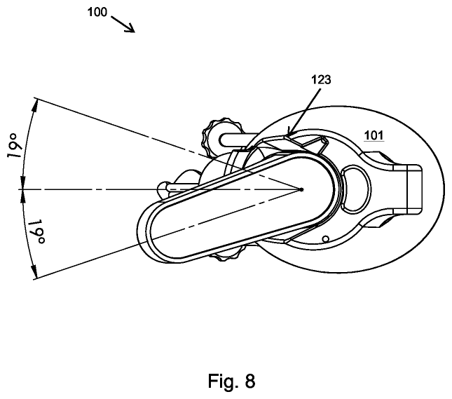

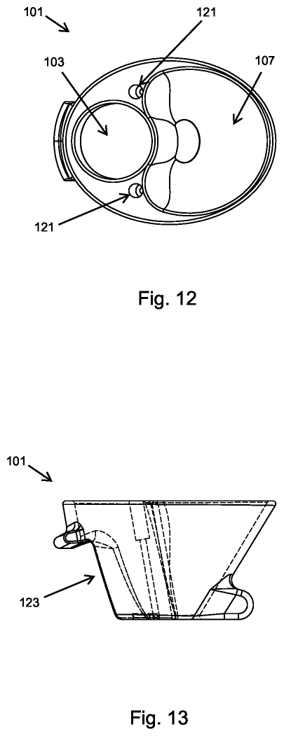

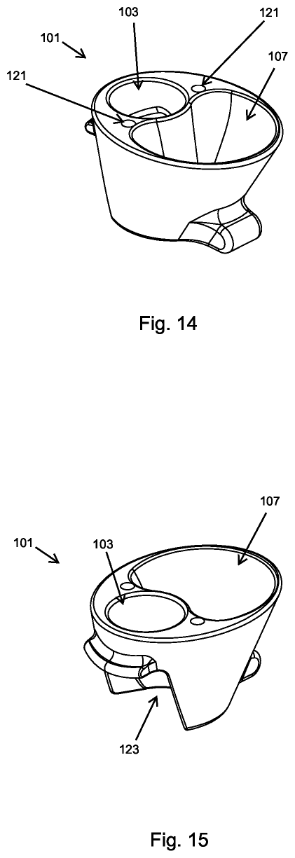

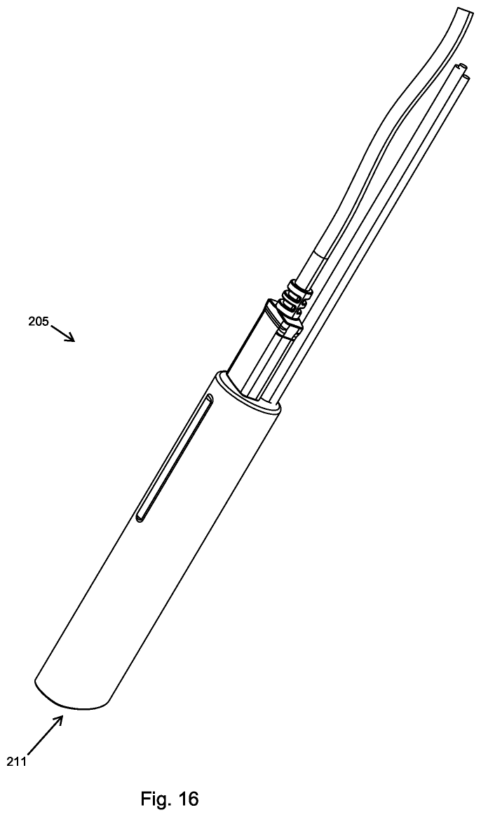

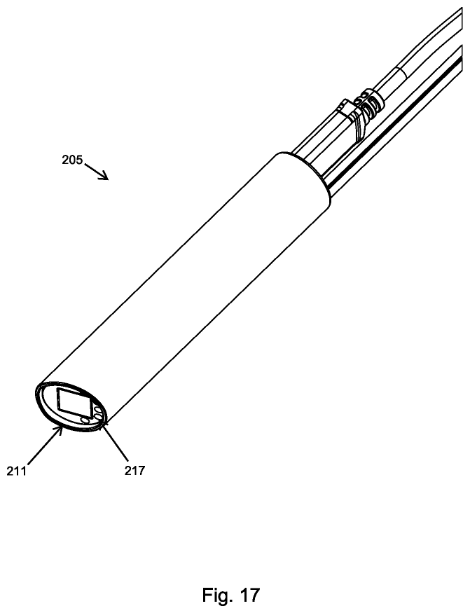

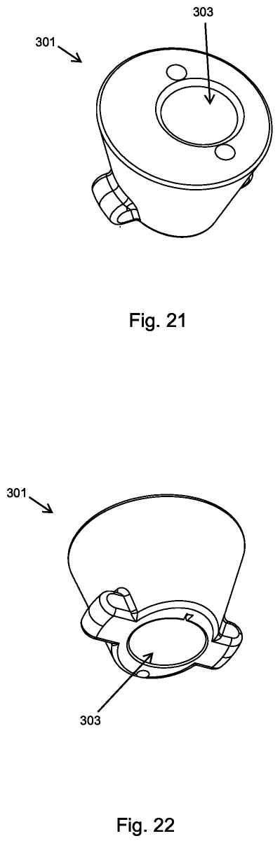

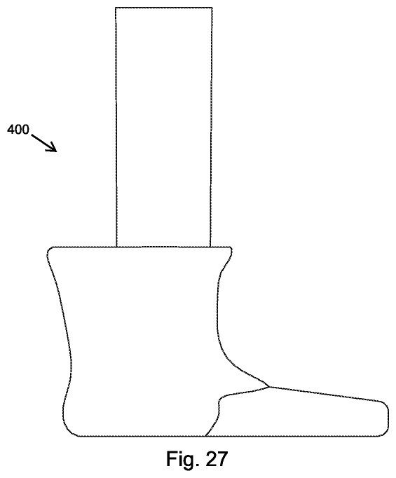

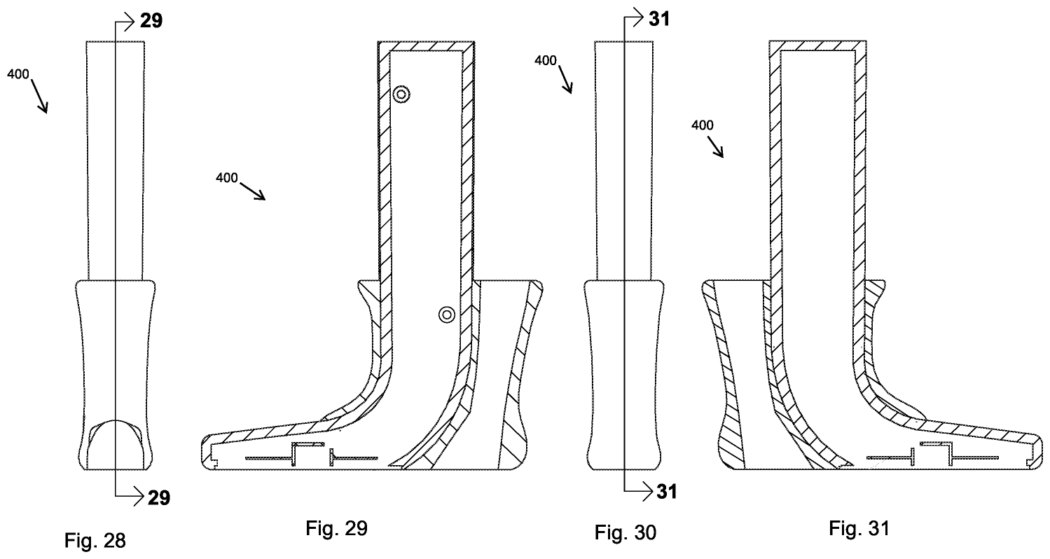

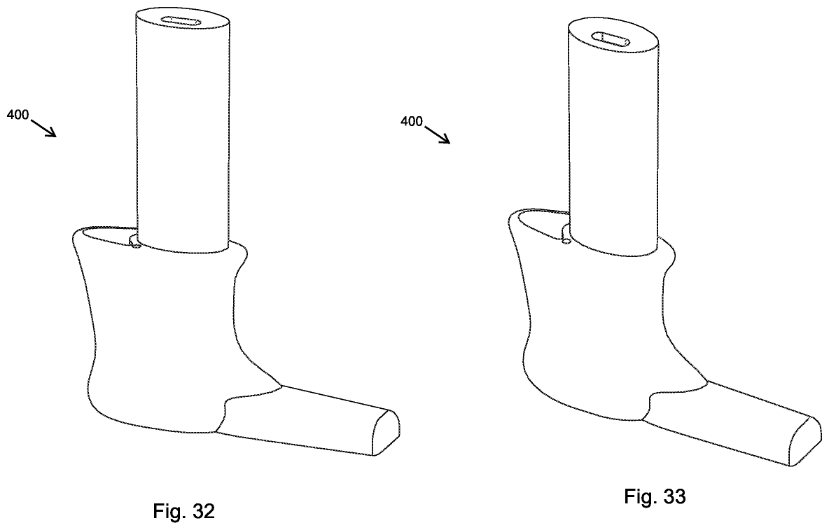

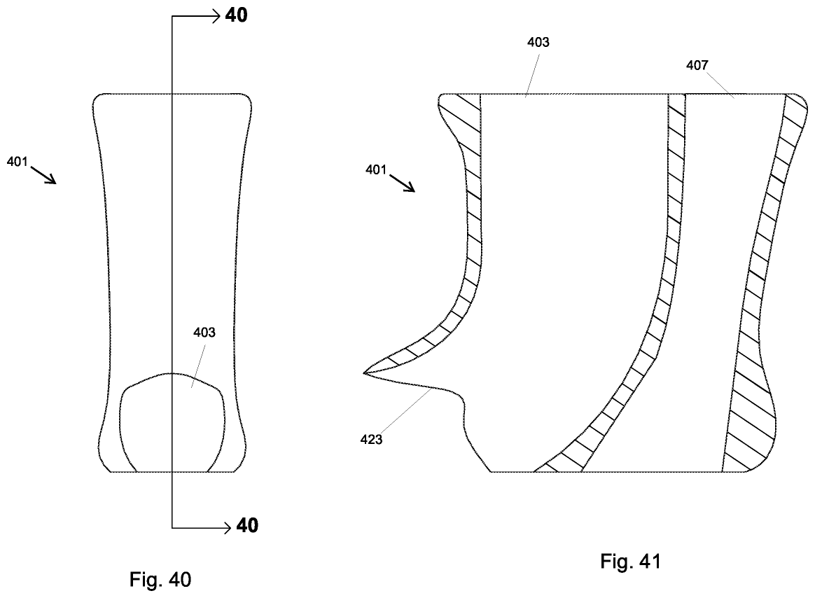

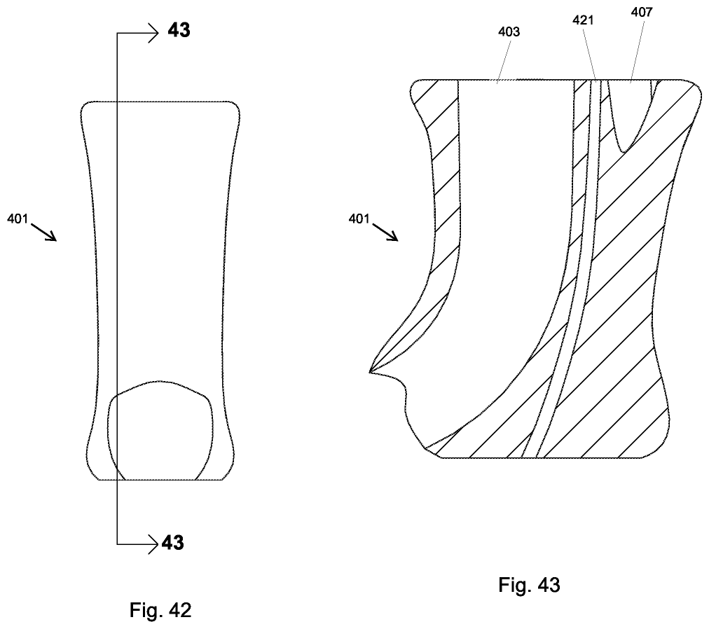

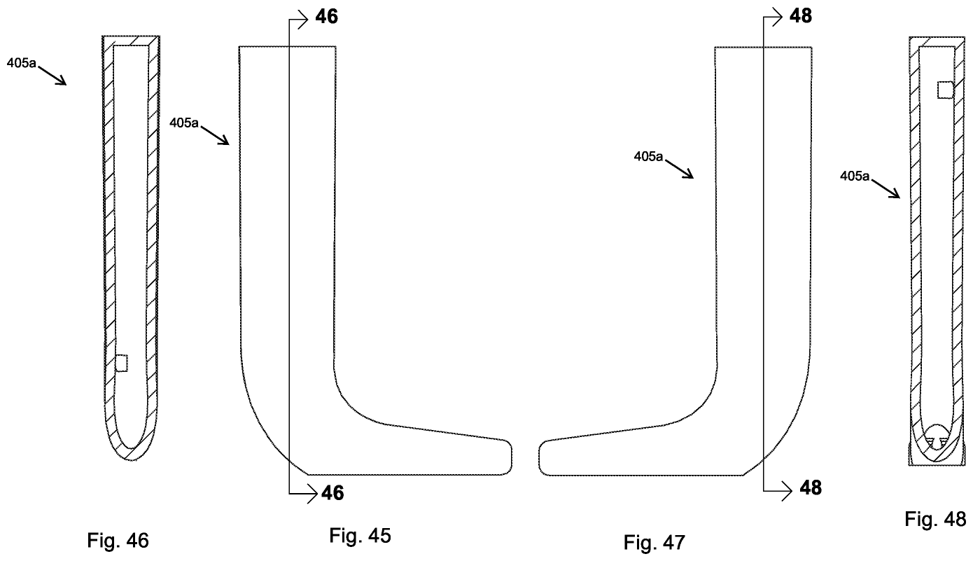

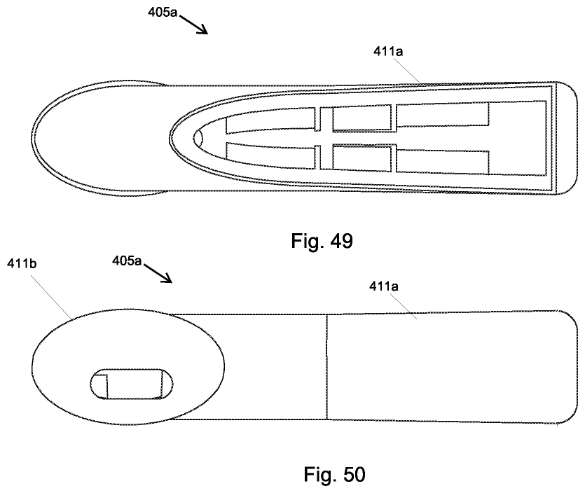

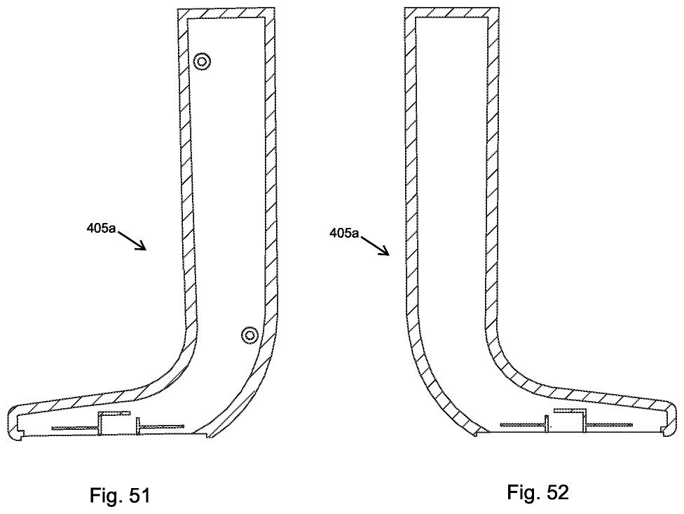

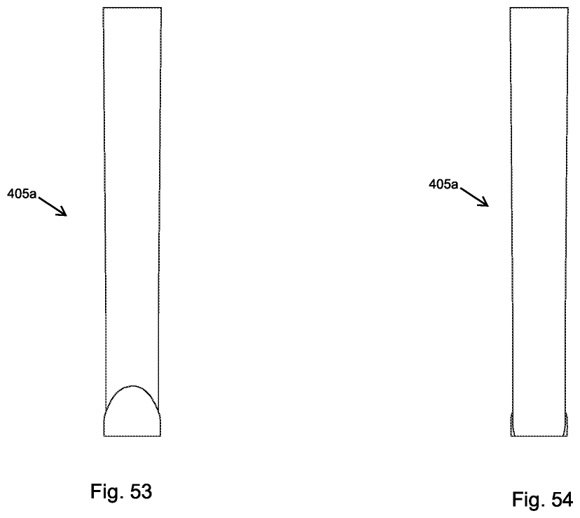

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1 and is designated generally by reference character 100. Other embodiments and/or aspects of this disclosure are shown in FIGS. 2-54.

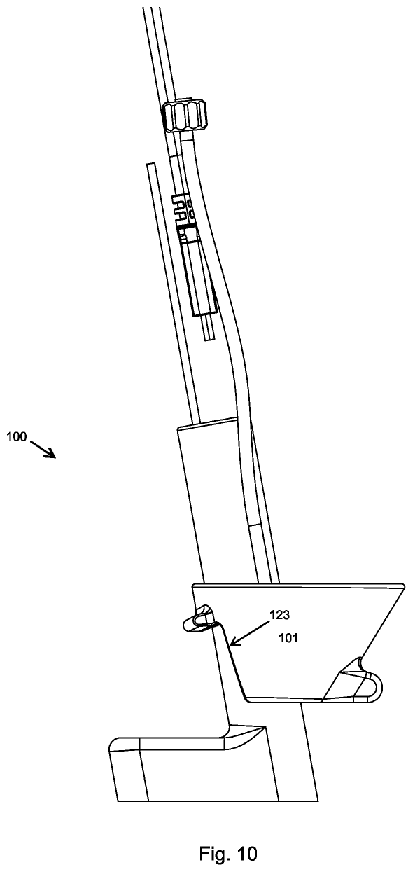

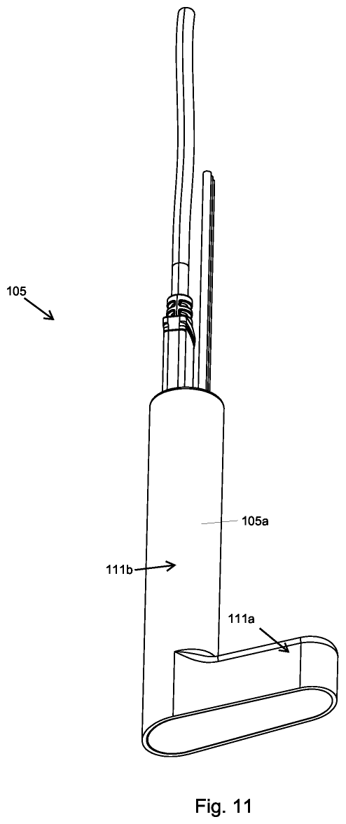

Referring to FIGS. 1-23 generally, and specifically to FIGS. 1-10, an access port system 100 can include a port body 101 configured to be inserted into an incision. Referring additionally to FIGS. 11-15, the port body 101 can define an imaging assembly opening 103 from a proximal side to a distal side thereof. The access port system 100 can include and an imaging assembly 105 configured to pass through the imaging assembly opening 103 to allow imaging of an interior portion of a patient body.

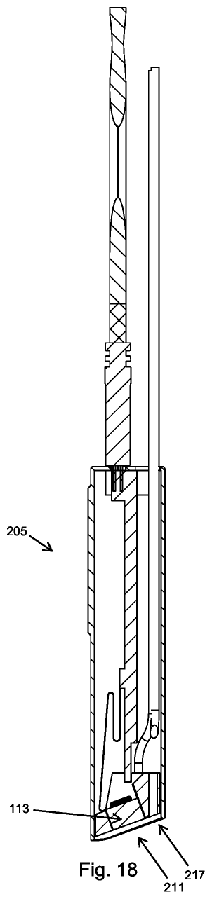

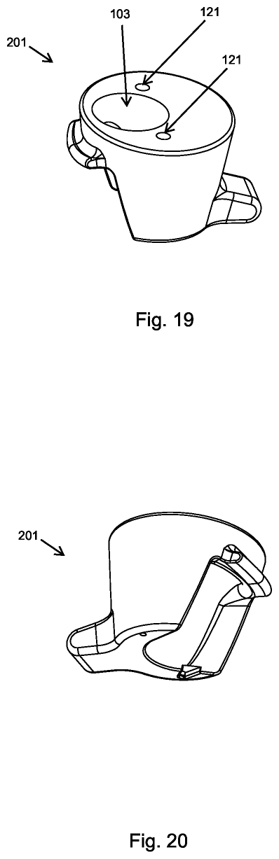

In certain embodiments, the port body 101 can be flexible (e.g., made of elastomeric material) and a housing 105a of the imaging assembly can be rigid (e.g., made of hard plastic or metal). In certain embodiments, the port body 101 can also define an instrument channel 107 from the proximal side thereof to the distal side to allow an instrument pass through to perform a medical procedure. In certain embodiments, e.g., as shown in FIGS. 19-22, the port body 201, 301 may not have an instrument channel, but only an imaging assembly opening 103, 303. The port body 201, 301 may be otherwise similar to the port body 101 as disclosed herein, or may be different in any suitable manner appreciated by those having ordinary skill in the art in view of this disclosure.

In certain embodiments, the port body 101 can include one or more anchors 109a, 109b extending from the port body 101. The one or more anchors 109a, 109b can include a heal anchor 109a adjacent the distal side of the port body 101, e.g., as shown. The one or more anchors 109a, 109b can include a toe anchor 109b disposed on an opposite side of the port body 101 relative to the heal anchor 109a and proximal of the heal anchor 109a, e.g., as shown.

In certain embodiments, referring to FIGS. 2 and 11, the imaging assembly 105 can include an imaging assembly radial portion 111a configured to house an imaging device 113 (e.g., a camera) disposed therein. The imaging assembly 105 can include an imaging assembly leg portion 111b extending proximally from the radial portion 111a (e.g., at an angle as shown). For example, the imaging assembly 105 can include a boot shape, e.g., as shown. In certain embodiments, the leg portion 111b can be angled at a non-right angle to the radial portion 111a.

The imaging assembly 105 can include an imaging device 113 located in the radial portion 111a at a radially outward position thereof, e.g., radially away from the leg portion 111b. A transparent layer 115 (e.g., made of glass or plastic) can be disposed on a distal side of the imaging device 113 (e.g., along the length of the radial portion 111a). The imaging assembly 105 can include one or more irrigation ports 117 (e.g., defied through the transparent layer 115) disposed proximate to the imaging device 113 to provide irrigation proximate the imaging device 113 (e.g., to clean the transparent layer 115 where the imaging device 113 is located).

The imaging assembly 105 can include one or more irrigation channels 119 connected to one or more irrigation ports 117. The one or more irrigation channels 119 can extend from the one or more irrigation ports 117, through the radial portion 111a, and through the leg portion 111b to a proximal end of the leg portion 111b (e.g., and exit therefrom), e.g., as shown. In certain embodiments, the housing 105a may include a data port (e.g., USB-C, USB3.0, or other suitable port) disposed at a proximal side thereof (e.g., shown having a data cable plugged in).

In certain embodiments, the port body 101 can include one or more insufflation 121 defined therethrough. The one or more insufflation ports 121 may include a tube connected or disposed therein, e.g., as shown in FIGS. 1-10.

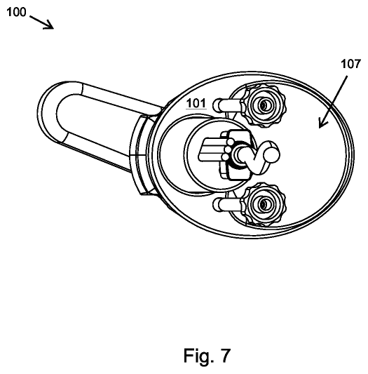

Referring to FIGS. 7-9, in certain embodiments, the port body 101 and the imaging assembly 105 can be configured such that the port body 101 allows the imaging assembly 105 to rotate within the port body 101. For example, the port body 101 can define a window 123 (e.g., a portion of the distal end of the imaging assembly opening 103) that limits a rotation of the radial portion 111a of the imaging assembly 105. For example, the window 123 can be sized limit rotation of the radial portion to about 25 degrees or less, e.g., about 19 degrees as shown. Any suitable limit is contemplated herein.

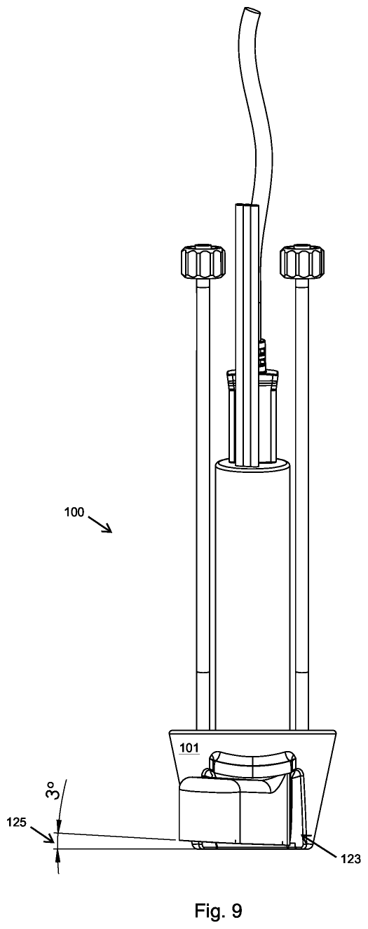

In certain embodiments, as shown in FIG. 9, the non-right angle between the leg portion 111b and the radial portion 111a causes a lifting angle 125 when rotated within the port body 101. For example, the lifting angle 125 can be about 3 degrees, e.g., as shown. Any suitable lifting angle 125, and any suitable non-right angle of the leg portion 111b is contemplated herein. In certain embodiments, e.g., as shown best in FIG. 2, the leg portion 111b can house imaging electronics (e.g., circuit board 127, data cable 129 to imaging device 113, and/or data port 131) and/or can act as a handle for rotation of the imaging assembly 105, for example. Any other suitable features are contemplated herein.

In certain embodiments, the non-right angle of the radial portion 111a between the rotatable axis and the vector of the radial extension of the radial portion 111a can be between about 30 degrees and about 120 degrees. The effect of decreasing the angle can cause an increase in the change of viewing angle. The angle of viewing can be further adjusted by the raising or lowering a slope of the lower surface of the radial extension from the tip to the leg portion 111b. Furthermore, the combination of variability of these characteristics can allow for the device to function facing any direction around an incision, for example.

Referring to FIGS. 16-18, in certain embodiments, instead of a boot shape for example, the imaging assembly 205 can be a straight member having an angled distal face 211. An imaging device 113 can be disposed at the angled distal face 211 to provide an angled view when inserted through the port body, e.g., 101, 201. The imaging assembly 205 can include one or more irrigation ports 217 disposed at the angled distal face 211 proximate the imaging device 113. The imaging assembly 205 can otherwise include any suitable features of the imaging assembly 105 fit into the straight housing (e.g., electronics, data port, irrigation channels, etc.). Any suitable software and/or hardware modules for imaging and/or performing any other procedure are contemplated herein.

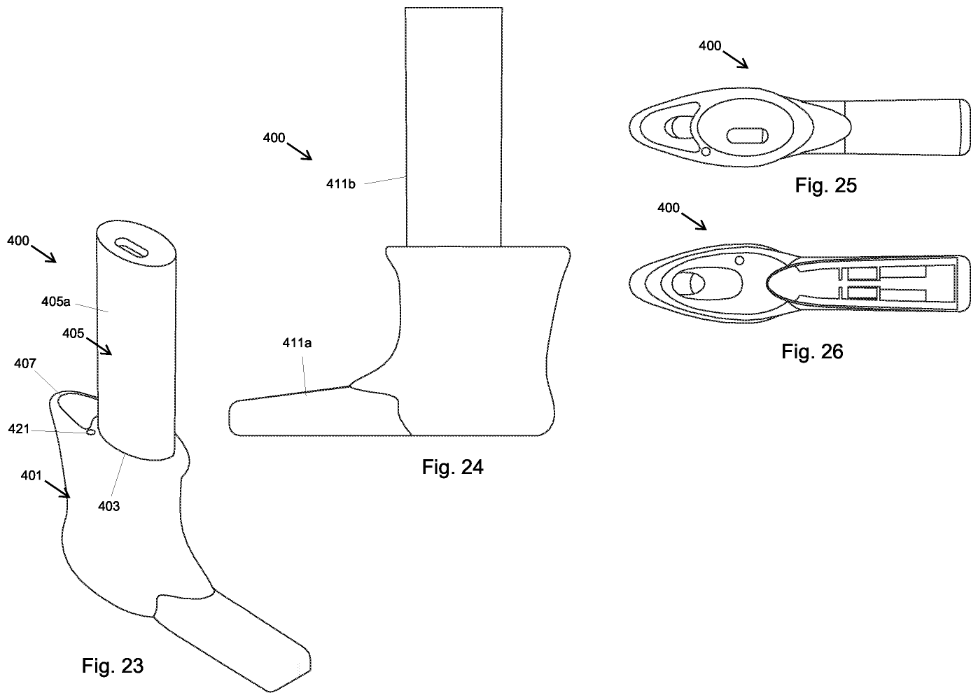

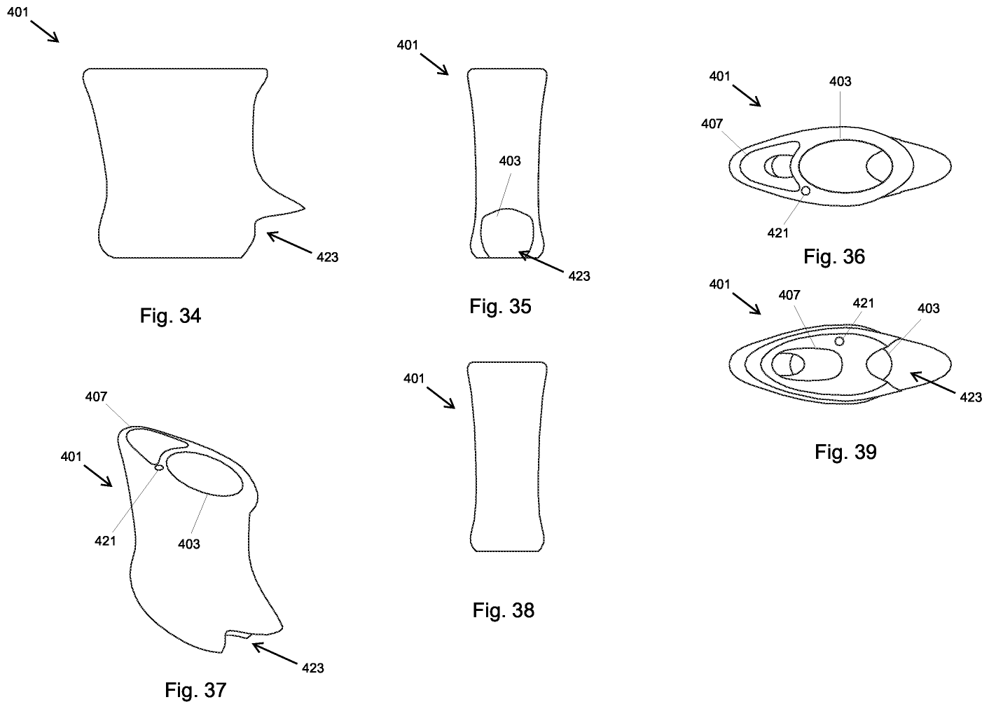

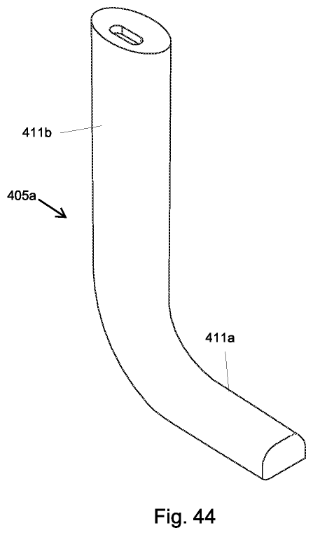

Referring to FIG. 23-33, another embodiment of an access port system 400 is shown in accordance with this disclosure. FIGS. 34-43 show an embodiment of a port body 401 of the embodiment of a system 400, shown in isolation. FIGS. 44-52 show an embodiment of an imaging assembly housing 405a of the embodiment of a system 400, shown in isolation.

The system 400 can be similar to the system 100 in certain embodiments. For example, the system can have a port body 401 that can be configured to be inserted into an incision, and the port body 401 can define an imaging assembly opening 403 from a proximal side to a distal side thereof.

The port body 401 can be similar to the port body 101, 201, 301 in certain embodiments. For example, the port body 401 can be made of a flexible material (e.g., silicone). The port body 401 can include an instrument channel 407 and an insufflation port 421. The port body 401 can include a swept back shape as shown, e.g., as opposed to a more rounded shape as shown for the port body 101, 201, 301. The swept back shape can allow for a smaller incision to be made.

The system 400 can include an imaging assembly 405 configured to pass through the imaging assembly opening 403 to allow imaging of an interior portion of a patient body (e.g., an abdomen location, e.g., a gallbladder area). The imaging assembly 405 can include an imaging assembly housing 405a. The imaging assembly housing 405a can include a similar overall shape as assembly 105 (e.g., a boot shape having a leg portion 411b and radial portion 411a). The imaging assembly housing 405a can have about a 90 degree angle between the radial portion 411a and the leg portion 411b. Any suitable angle is contemplated herein.

The imaging assembly 405 can include similar components (e.g., imaging devices, cables, etc.) and in similar locations to the imaging assembly 105 for example. For example, the imaging assembly 405 can include an imaging device and/or lighting in the radial extension 411a (e.g., a locations. One or more cables (e.g., a MIPI cable) can travel through the interior cavity of the imaging assembly housing 405a to a location to be connected. A transparent layer (not shown, similar to layer 115), e.g., made of glass or plastic, can be disposed on a distal side of the imaging device (e.g., along the length of the radial portion 411a).

As shown, the imaging assembly housing 405a can include a swept back shape. For example, the imaging assembly housing can have an oval cross-sectional shape. Such a swept back shape can allow for a reduction in the width of the entire system 400 including the port body 401. For example, as shown, the imaging assembly opening 403 can include a complimentary shape to the imaging assembly housing 405a to create a suitable seal. Any suitable shape to allow for a seal is contemplated herein. As disclosed above, the port body 401 can also include a swept back shape aiding in reduction of overall width and reduction in incision size, for example. Any other suitable form factor for reducing incision size is contemplated herein. In certain embodiments, the components of system 400 can be sized to allow for an 8 mm incision or smaller.

The port body 401 can define a window 423 (e.g., a portion of the distal end of the imaging assembly opening 403) where the radial extension 411a of the imaging assembly 405 extends from. For example, the window 123 can be sized to seal against the radial extension 411a. As shown, the oval shape for the imaging assembly housing 405a and complimentary shaped opening 403 can prevent rotation of the housing 405a relative to the port body 401, and a wider window with clearance is not necessary for the system 400. As shown, the imaging assembly 405 can be fixed in relative rotational position to the port body 401. The port body 401 and/or the opening 403 can be shaped to prevent rotation of the imaging assembly 405, unlike the embodiment of FIG. 1. The port body 401 can include any suitable openings, ports, etc. as appreciated by those having ordinary skill in the art in view of this disclosure (e.g., as described above with respect to other embodiments).

The imaging assembly housing 405a can be made of a rigid material in certain embodiments. The imagine assembly housing 405a can be inserted through the imaging assembly opening 403 (e.g., which is made easier by the port body 401 being compliant).

Once assembled, the system 400 can be inserted into an incision (e.g., in a shoehorning motion). The area can be insufflated if desired, e.g., through port 421. Images can be received from the imaging device in the radial extension 411a and the surgical area can be viewed. The surgical site can be accessed via the instrument channel 407 while viewing the area.

Certain embodiments of this disclosure can employ one or more imaging devices, e.g., as disclosed above. Certain image processing can be conducted on data received from the imaging device. For example, in the embodiment of a system 400, where the imaging assembly 405 may be fixed relative to the port body 401, no mechanical movement of the imaging device may be possible. In fact, certain embodiments, e.g., system 400, may have no moving parts at all. In view of this, embodiments can include an image processing module configured to allow digital movement of an image in situ. For example, where a resolution of a camera is sufficiently high (e.g., 4K), no magnification may be needed (e.g., at the distances of an abdominal procedure using insufflation). The resolution of the surgical site with an unmagnified camera of suitable resolution can be equivalent or better than a traditional endoscope provides. In such embodiments, the image processing module (which can be located internal or external to the system 400), can receive one or more commands from a user to provide a digital zoom and/or pan of the images/video stream.

The image processing module can be configured to connect to a controller (e.g., a dual joystick controller and/or any other suitable controller) to receive the commands. For example, one stick can control zoom, and another stick can control vertical and lateral pan. The image processing module can be configured to output the processed images/video stream to a screen for the user to view in real time. Any suitable input mechanism for digital image pan and/or zoom is contemplated herein. Such a control mechanism eliminates the need to learn reverse control of an endoscope in use, speeds up target image acquisition, and reduces chances for error. Such control schemes also allow elimination of moving parts and a wider field of view overall. Any other suitable additional image processing (e.g., dewarping to create a flat image, 3D image creation using different flashing light positions to provide differing shadows, etc.) are contemplated herein.

As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. An access port system, comprising:
a port body configured to be inserted into an incision, the port body defining an imaging assembly opening from a proximal side to a distal side thereof; and
an imaging assembly configured to pass through the imaging assembly opening to allow imaging of an interior portion of a patient body.

2. The system of claim 1, wherein the port body is flexible and a housing of the imaging assembly is rigid.

3. The system of claim 1, wherein the port body also define an instrument channel from the proximal side thereof to the distal side to allow an instrument pass through to perform a medical procedure.

4. The system of claim 1, wherein the port body includes one or more anchors extending from the port body.

5. The system of claim 4, wherein the one or more anchors include a heal anchor adjacent the distal side of the port body.

6. The system of claim 5, wherein the one or more anchors includes a toe anchor disposed on an opposite side of the port body relative to the heal anchor and proximal of the heal anchor.

7. The system of claim 1, wherein the imaging assembly includes a radial portion configured to house an imaging device disposed therein and a leg portion extending proximally from the radial portion.

8. The system of claim 7, wherein the imaging assembly includes a boot shape.

9. The system of claim 8, wherein the leg portion is angled at a right angle to the radial portion.

10. The system of claim 7, wherein the imaging assembly includes an imaging device located in the radial portion at a radially outward position thereof.

11. The system of claim 1, wherein the port body includes an instrument channel defined therethrough from a proximal side to a distal side thereof.

12. The system of claim 11, wherein the port body includes an insufflation port defined therethrough from a proximal side to a distal side thereof.

13. The system of claim 12, wherein the port body is made of silicone.

14. The system of claim 7, wherein the port body and the imaging assembly are configured such that imaging assembly does not rotate within the port body when inserted into the port body.

15. The system of claim 14, wherein the port body defines a window that seals to the radial portion of the imaging assembly.

16. The system of claim 1, wherein the port body includes a swept back shape.

17. The system of claim 16, wherein an imaging device housing includes a swept back shape.

18. The system of claim 17, wherein the imaging device housing includes an oval cross-sectional shape.

19. The system of claim 1, wherein the imaging assembly is a straight member having an angled distal face, wherein an imaging device is disposed at the angled distal face to provide an angled view when inserted through the port body.

20. The system of claim 1, wherein the system further comprises an image processing module configured to allow digital movement of an image in situ.
